(19) United States
(12) Patent Application Publication
(10) Pub. No.: US 2016/0116472 A1
(43) Pub. Date: Apr. 28, 2016

(54) BIOMARKERS FOR STROKE DIAGNOSIS

(71) Applicant: THE GENERAL HOSPITAL CORPORATION, Boston, MA (US)

(72) Inventor: Hakan Ay, Arlington, MA (US)

(73) Assignee: The General Hospital Corporation, Boston, MA (US)

(21) Appl. No.: 14/764,722

(22) PCT Filed: Feb. 4, 2014
(86) PCT No.: PCT/US2014/014579

§ 371 Date: Jul. 30, 2015

Related U.S. Application Data
(60) Provisional application No. 61/760,324, filed on Feb. 4, 2013.

(51) Int. Cl. G01N 33/573; A61B 5/00; A61B 5/055

(52) U.S. Cl. CPC G01N 33/573; A61B 5/055; A61B 5/4064; G01N 2333/91102; G01N 2800/2871

(57) ABSTRACT

Disclosed herein are assays and methods of diagnosing strokes using glycogen phosphorylase-BB (GPBB) as the biomarker. Specifically, assays and methods are described herein for distinguishing an ischemic brain condition from myocardial infarction, or distinguishing an ischemic brain condition from stroke mimics, or distinguishing an ischemic stroke from a hemorrhagic stroke. The diagnostic results offered by these assays and methods allow early and appropriate treatment for the specific condition. Also disclosed is a method to identify whether a subject having an ischemic stroke exhibits salvageable brain tissue.

### CROSS-REFERENCE TO RELATED APPLICATION

This application claims benefit under 35 U.S.C. §119(e) of U.S. Provisional Application No. 61/760,324 filed Feb. 4, 2013, the contents of which are incorporated herein by reference in its entirety.

### TECHNICAL FIELD

The present disclosure relates generally to biomarkers for diagnosis of strokes.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Strokes are sudden neurological disorders that occur when blood flow to the brain is disturbed. There are two kinds of strokes. One is called acute ischemic stroke (AIS), which is due to blood flow blockage, and it accounts for 87% of strokes. The other kind is called hemorrhagic stroke, which is caused by a weakened blood vessel that ruptures and bleeds into the surrounding brain tissue. Symptoms of strokes include sudden numbness or weakness of the face, arm, or leg, sudden confusion, dizziness, and sudden headache.

Strokes can lead to loss of brain functions or even death. Statistics shows that about 20% of those who have a stroke die in the hospital. This proportion is even higher among elderly people. Earlier treatment for stroke patients gives better outcomes. Optimally, stroke patients must be treated within the first three hours of symptom onset to reduce brain damage. Because time is critical for stroke patients, rapid diagnosis of strokes is highly desired. Currently, a physician diagnoses a stroke in a patient by medical history, physical exam, and neuroimaging (e.g., magnetic resonance imaging (MRI) or computerized tomography). In particular, neuroimaging requires highly trained technicians and sophisticated instrumentations. In addition, accurate diagnosis is critical for proper treatment, as different stroke causes require different treatments. For example, a patient suffering from an AIS would need a different treatment compared to a patient suffering from a hemorrhagic stroke. However, stroke diagnosis is complicated by the fact that not only are there different kinds of strokes, but there are other ischemic and non-ischemic conditions mimicking AIS. For example, an ischemic condition mimicking AIS can be a transient ischemic attack (TIA), in which a temporary interruption in blood flow to part of the brain results in impaired brain functions, but does not necessarily result in brain tissue damage. A non-ischemic condition mimicking AIS or TIA is not a stroke, but it can exhibit the same symptoms as a stroke, thus making it challenging for a physician to make an accurate diagnosis. A non-ischemic condition mimicking AIS or TIA can include, but is not limited to, migraine, hypoglycemia, and brain tumor.

### SUMMARY

The following embodiments and aspects thereof are described and illustrated in conjunction with systems, compositions and methods which are meant to be exemplary and illustrative, not limiting in scope.

Described herein are biomarkers, methods, and compositions, for improved accuracy and timing in the diagnosis of strokes. By studying the glycogen phosphorylase-BB (GPBB) levels in patients suspected of suffering from a stroke, the inventors have surprisingly discovered that the level of GPBB is elevated in patients with an ischemic brain condition such as acute ischemic stroke (AIS) or transient ischemic attack (TIA). In addition, the size of ischemic brain tissue (live or dead) is correlated with the level of GPBB. Accordingly, the present invention establishes that GPBB is a sensitive biomarker for early stroke diagnosis, and GPBB can be used in a simple blood test to help diagnose strokes.

In one aspect, described herein is an assay comprising measuring a level of GPBB and a level of troponin in a sample obtained from a subject, comparing the level of GPBB with a first reference level and the level of troponin with a second reference level, and if the level of GPBB is at or above the first reference level and if the level of troponin is below the second reference level, identifying the GPBB as originating from the subject's brain.

In another aspect, described herein are an assay and a method for diagnosing an ischemic brain condition in a subject, comprising measuring a level of GPBB in a sample obtained from the subject, and identifying the subject as having (a) an AIS or a TIA if the level of GPBB is at or above a reference level, or (b) a non-ischemic condition mimicking AIS or TIA if the level of GPBB is below the reference level. In some embodiments, treatment based on the level of GPBB and appropriate for the identified condition is provided to the subject.

In some embodiments, the sample is a blood, serum or plasma sample.

In some embodiments, the level of GPBB is measured by an immunoassay (e.g., ELISA).

In another aspect, described herein are an assay and method of distinguishing brain ischemia from a heart condition in a subject, the method comprises assaying a level of GPBB in a sample obtained from the subject, assaying a level of troponin in a sample obtained from the subject, comparing the level of GPBB with a first reference level, comparing the level of troponin with a second reference level, and identifying the subject as having brain ischemia, e.g., when the troponin level is below the second reference level and the GPBB level is at or above the first reference level.

In some embodiments, the level of troponin is measured by an immunoassay.

In some embodiments, the troponin is troponin T, troponin I, or a combination thereof.

Also described herein is a substrate composition comprising a first binding agent which specifically binds to GPBB and a second binding agent which specifically binds to troponin.

In some embodiments, the first binding agent comprises an antibody that specifically binds to GPBB, and the second binding agent comprises an antibody that specifically binds to troponin.

One aspect described herein relates to a method of diagnosing stroke in a subject with one or more stroke symptoms, the method comprises contacting a sample obtained from the subject with a substrate composition as described herein, and detecting the amounts of GPBB and troponin in the sample, wherein the amounts permit diagnosis of stroke.

A further aspect of the invention relates to an assay and a method of distinguishing an AIS from a hemorrhagic stroke, the method comprising (i) measuring, in a sample obtained from a subject experiencing a stroke, a level of GPBB; (ii) comparing the level of GPBB with a reference level; and identifying the subject as having an AIS if the level of GPBB is at or above a reference level, or a hemorrhagic stroke if the level of GPBB is below the reference level. In some embodiments, a treatment is provided to the subject based on the identification of the stroke as ischemic or hemorrhagic. For example, if the subject is identified as having an AIS, an antithrombotic agent is administered to the subject, and if the subject is identified as having a hemorrhagic stroke, an antithrombotic agent is not administered to the subject.

Another aspect relates to a method of treating a subject exhibiting one or more stroke symptoms, the method comprising assaying a level of GPBB in a sample obtained from the subject, comparing the level of GPBB with a reference level, identifying the subject as having an AIS if the level of GPBB is at or above the reference level, and if the subject is identified as having and AIS, administering a treatment for AIS to the subject.

Relatedly, one aspect regards a method of selecting a patient for treatment, the method comprising assaying a level of GPBB in a sample obtained from a subject or patient, comparing the level of GPBB with a reference level, determining whether the patient has an AIS or TIA based on the comparison, and providing a treatment appropriate for the patient's condition.

A further aspect regards a method of classifying or labeling a drug or drug dosage form for treatment of central nervous system (CNS) indications, the method comprising classifying or labeling the drug or drug dosage form according to the GPBB level in a sample from a patient in need of the drug or drug dosage form. In some embodiments, the drug or drug dosage form is classified or labeled such that if the GPBB level of the patient is at or above a reference level, the drug or dosage form is to be administered for a CNS indication, and if the GPBB level is below the reference level, then the drug or drug dosage form is classified or labeled against administration for that CNS indication. In some embodiments, the CNS indication is an AIS or TIA, and the drug or drug dosage form is an antithrombotic.

The GPBB level can be used to monitor treatment progress or the extent of ischemic brain injury. One aspect of the technology described herein relates to a method of monitoring treatment progress in a subject suffering from an ischemic stroke, the method comprising: (1) measuring at a first time point, a first level of GPBB in a first sample obtained from the subject; (2) measuring at a second time point, a second level of GPBB in a second sample obtained from the subject, wherein the second time point is later than the first time point and after the administration of a therapeutic agent for ischemic stroke, and wherein if the second level of GPBB is lower than a reference value set for the second time point, then the treatment is considered to be effective.

Another aspect regards a method of monitoring an extent of ischemic brain injury in a subject who has an AIS or a TIA, the method comprising: measuring, at two or more time points, levels of GPBB from samples obtained from the subject, wherein an increase in the level of GPBB indicates a likelihood of expansion of ischemic brain injury.

A further aspect regards a method of identifying whether a subject with an AIS or a TIA exhibits salvageable brain tissue (i.e., ischemic but viable brain tissue or ischemic penumbra), the method comprising measuring a level of GPBB in a sample obtained from the subject, identifying a size or amount of irreversibly injured brain tissue based on neuroimaging, correlating the size of irreversibly injured brain tissue with an expected GPBB level, and if the level of GPBB is above the expected GPBB level, identifying the subject as having salvageable brain tissue.

In some embodiments, the neuroimaging is magnetic resonance imaging.

In some embodiments, the neuroimaging is a computed tomography scan.

In some embodiments, the subject is a human.

### BRIEF DESCRIPTION OF THE DRAWINGS

Exemplary embodiments are illustrated in referenced FIGURES. It is intended that the embodiments and FIGURES disclosed herein are to be considered illustrative rather than restrictive.

FIG. 1 is a plot of plasma GPBB levels in 172 patients. Crosses indicate controls, and open circles indicate cases. The dash line is a reference level.

### DETAILED DESCRIPTION

All references cited herein, including the references cited therein, are incorporated by reference in their entirety as though fully set forth. Unless defined otherwise, technical and scientific terms used herein have the same meaning as commonly understood by one of ordinary skill in the art to which this invention belongs. The practice of the present invention will employ, unless indicated specifically to the contrary, conventional methods of molecular biology and recombinant DNA techniques within the skill of the art, many of which are described below for the purpose of illustration. Such techniques are fully explained in the literature. See, e.g., Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001), Sambrook, et al., Molecular Cloning: A Laboratory Manual (3rd Edition, 2000); Sambrook and Russel, Molecular Cloning: A Laboratory Manual 3rd ed., Cold Spring Harbor Laboratory Press (Cold Spring Harbor, N. Y. 2001), DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005), Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, (2002, Cold Spring Harbor Laboratory, ISBN 0-87969-630-3) provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

A blood test for stroke is highly desired in stroke diagnostics. Several potential biomarkers have been studied in ischemic strokes, yet none has reached the sensitivity and specificity required for routine clinical use.

Primarily found in the brain and heart, glycogen phosphorylase-BB (GPBB) is the key enzyme that breaks down glycogen to yield glucose-1-phosphate in response to ischemia. By studying the GPBB levels in patients suspected of suffering from a stroke, the inventor has surprisingly discovered that the level of GPBB is elevated in patients with an ischemic brain condition such as acute ischemic stroke (AIS) or transient ischemic attack (TIA). Without wishing to be bound by theory, during brain ischemia, the supply of energy to the brain is reduced at least locally, so a surge in GPBB level is needed to meet the emergent energy need (in the form of adenosine triphosphate) in the brain. Accordingly, the present invention establishes that GPBB is a sensitive biomarker for early stroke diagnosis, and therefore provides rapid and accurate diagnostic methods that answer the following critical clinical questions in diagnosing a stroke: (1) Is this a stroke or a stroke mimic? And (2) If this is a stroke, is this an AIS or a hemorrhagic stroke?

It should be noted that the assays and methods described herein can be used together with one or more other diagnostic methods for strokes, including, but not limited to, neuroimaging (e.g., magnetic resonance imaging (MRI), computerized tomography (CT), diffuse optical imaging, event-related optical signal, functional MRI, magnetoencephalography, positron emission tomography, or single-photon emission computed tomography), medical history, and physical exam.

One aspect of the invention provides an assay and a method for diagnosing an ischemic brain condition in a subject, comprising measuring a level of GPBB in a sample obtained from the subject, and identifying the subject as having (a) an AIS or a TIA if the level of GPBB is at or above a reference level, or (b) a non-ischemic condition mimicking AIS or TIA if the level of GPBB is below the reference level.

In some embodiments, the sample is a blood, serum or plasma sample.

In some embodiments, the sample is obtained following the onset of one or more stroke symptoms. Stroke symptoms can include, but are not limited to, sudden numbness, tingling, weakness, or loss of movement in the face, arm, or leg, especially on only one side of the body; sudden vision changes; sudden trouble speaking; sudden confusion or trouble understanding simple statements; sudden problems with walking or balance; a sudden, severe headache that is different from past headaches.

In some embodiments, the sample is obtained during the persistence of one or more stroke symptoms.

It is contemplated that the sample could be obtained when a subject is experiencing a silent stroke. A silent stroke is a stroke that does not have any outward stroke symptoms, and the subject typically is unaware of the stroke happening.

### Brain Ischemia

Brain ischemia, also known as cerebral ischemia, is a neurological condition in which the blood flow to the brain becomes insufficient to meet metabolic demand. Prolonged brain ischemia can lead to the death of brain tissue due to limited oxygen and nutrient supply. Conditions resulting from brain ischemia include, but are not limited to AIS and TIA.

AIS is an episode of neurological dysfunction caused by focal brain, spinal cord, or retina ischemia with evidence of acute infarction (Easton et al., Stroke 2009, 40, 2276-2293). There are at least four different causes of blood flow interruption: (1) a blood clot in a blood vessel; (2) a blood clot in the dural venous sinuses, which drain blood from the brain; (3) an embolus clogging a blood vessel; or (4) a sudden drop in blood pressure. Stroke symptoms can and frequently do persist beyond 24 hours if the patient survives the initial damage.

TIA, also called a mini-stroke, is a transient episode of neurological dysfunction caused by focal brain, spinal cord, or retinal ischemia without evidence of acute infarction. TIA symptoms can initially be the same as a stroke, except that the symptoms only last a short time, typically within 2-15 minutes, or at most 24 hours. Even though a TIA is temporary and usually does not cause brain tissue damage, a patient experiencing TIA is advised to seek professional help immediately because of the similarity in symptoms and because TIA is a risk factor for subsequent ischemic strokes.

In some embodiments, the assays and methods described herein can aid in distinguishing an AIS or TIA from a non-ischemic condition mimicking AIS or TIA. A non-ischemic condition mimicking AIS or TIA, also called a stroke mimic, can exhibit some or all of the same symptoms as a stroke, except that it is not a stroke and it is not a result of brain ischemia. Without wishing to be bound by theory, because an elevated GPBB level is the result of an ischemic condition, a subject having a stroke mimic will not have an elevated GPBB level. Common stroke mimics include, but are not limited to, systemic infection, brain tumors, hypoglycemia, hyponatremia, complex migraine, and seizure. Less common stroke mimics include, but are not limited to, conversion disorder, vestibular dysfunction, cardiac, hypotension, trauma, subdural hematoma, herpes encephalitis, transient global amnesia, dementia, acute mono-neuropathy, demyelinating disease, spinal cord injury or lesion, myasthenia gravis, Parkinsonism, and hypertensive encephalopathy.

One study found a high rate of misdiagnosis among patients having stroke mimics: about 19%, or about 1 in 5 patients diagnosed with AIS by neurologists before cranial computed tomography (CT) scanning actually had non-cerebrovascular causes for their symptoms. Therefore, the assays and methods described herein can help reduce the high rate of misdiagnosis.

Without wishing to be bound by theory, the GPBB level in a sample obtained from a subject is correlated with the volume of injured brain tissue. Injured brain tissue includes infarction (i.e., dead tissue or tissue with irreversible injury) and ischemic but viable brain tissue (i.e., ischemic penumbra), each of which is responsible for elevating the GPBB level. The ischemic but viable brain tissue is salvageable and clinically important. However, standard neuroimaging techniques such as MRI or CT scanning are still problematic in imaging the ischemic but viable brain tissue. Even though positron emission tomography (PET) can image the ischemic but viable brain tissue, PET is neither widely available nor rapidly accessible.

Accordingly, another aspect of the technology disclosed herein relates to a method of identifying whether a subject with an AIS or a TIA exhibits salvageable brain tissue (i.e., ischemic but viable brain tissue), by combining measuring GPBB level with neuroimaging. By performing neuroimaging such as MRI or CT scanning on the subject, a physician can properly identify the size of apparent irreversibly injured brain tissue. The size of apparent irreversibly injured brain tissue can be correlated with an expected GPBB level. Without wishing to be bound by theory, a larger size of irreversibly injured brain tissue is correlated with a higher GPBB level. If the measured GPBB level is higher than the expected GPBB level, it indicates that the subject has salvageable brain tissue, even though the salvageable brain tissue does not show up in neuroimaging. The salvageable brain tissue can be surrounding the irreversibly injured brain tissue, or within the irreversibly injured brain tissue, or in a different part of the brain from the irreversibly injured brain tissue. In some embodiments, apparent irreversibly injured brain tissue can include salvageable brain tissue. In some embodiments, apparent irreversibly injured brain tissue does not include salvageable brain tissue. In some embodiments, the expected GPBB level can be a level of GPBB in a subject exhibiting the same size of irreversibly injured brain tissue. In some embodiments, the expected GPBB level can be the average level of GPBB in a pool of subjects exhibiting the same size of irreversibly injured brain tissue. In some embodiments, the expected GPBB level can be derived from a mathematical fit from the correlation of GPBB level with the size of irreversibly injured brain tissue.

The steps of the method comprises measuring a level of GPBB in a sample obtained from the subject, identifying a size of apparent irreversibly injured brain tissue based on neuroimaging, correlating the size of apparent irreversibly injured brain tissue with an expected GPBB level, and if the level of GPBB is above the expected GPBB level, identifying the subject as having salvageable brain tissue. In some embodiments, the level of GPBB is at least 10% higher than the expected GPBB level. In some embodiments, the level of GPBB is at least 20% higher than the expected GPBB level. In some embodiments, the level of GPBB is at least 30% higher than the expected GPBB level. In some embodiments, the level of GPBB is at least 40% higher than the expected GPBB level. In some embodiments, the level of GPBB is at least 100% higher than the expected GPBB level. In some embodiments, the level of GPBB is at least 200% higher than the expected GPBB level.

Yet another aspect of the technology disclosed herein relates to a method of monitoring the extent of ischemic brain injury in a subject who has AIS or a TIA. By monitoring the GPBB levels at 2, 3, 4, 5, 6, 7, 8, 9, 10 or more time points, if the GPBB level increases over time, it indicates a likelihood of expansion of ischemic brain injury.

GPBB from Myocardial Ischemia

Since myocardial ischemia can also contribute to an elevated level of GPBB in the blood, an elevated level of GPBB is not a definitive indication of brain ischemia in a subject.

In some embodiments, a method is performed to determine whether a subject suspected of having a stroke has myocardial ischemia (e.g., myocardial infarction).

In some embodiments, a biomarker specific for myocardial ischemia is used to help determine the origin of the elevated level of GPBB. If the biomarker specific for myocardial ischemia indicates that the subject does not have myocardial ischemia, then the elevated level of GPBB is due to an ischemic brain condition (i.e. AIS or TIA). If the biomarker specific for myocardial ischemia indicates that the subject has myocardial ischemia, then the subject might or might not be experiencing an ischemic brain condition. In this scenario, other diagnostic methods are needed to determine whether the subject has AIS or TIA. Biomarkers for myocardial ischemia include, but are not limited to troponins, ischemia-modified albumin, B-type natriuretic peptide (BNP), NT-proBNP, whole blood choline, and unesterified free fatty acid. Some biomarkers for diagnosing myocardial ischemia are disclosed in U.S. Pat. No. 8,497,078, US20130330744, US20130236917, US20080261317, WO2012122094, the content of each of which is incorporated by reference for its entirety.

It should be understood that essentially any such marker for myocardial ischemia can be used to assist the determination of the likely source of an elevated GPBB level. In some embodiments, the biomarkers specific for myocardial ischemia are cardiac troponins. For example, the inventors have shown that they can exclude diagnostic error arising from potential leaks of GPBB from the heart, by assaying for troponin in conjunction with GPBB (Example 2). Cardiac troponins are the gold standard for the diagnosis of acute myocardial infarction. Cardiac troponins applicable for the invention can include, but are not limited to troponin T and troponin I (Collinson et al., Ann. Clin. Biochem. 2001, 38, 423-49; Zethelius et al., Circulation 2006, 113, 1071-8). Methods of measuring the levels of cardiac troponins are well known in the art. For example, cardiac troponins can be measured using immunoassays, such as the Roche Elecsys Troponin T high sensitivity (cTnT hs) assay and cTnI immunoassays. Hence, another aspect of the technology disclosed herein regards an assay and a method of distinguishing brain ischemia from a heart condition in a subject, comprising measuring both the levels of GPBB and troponin, and after comparing the GPBB level and troponin level with their respective reference levels, if the troponin level is negative (i.e., at or below a normal reference level for troponin), and if the GPBB level is positive (i.e., at or above the normal reference level for GPBB), then the subject is identified as having brain ischemia. It should be understood that the troponin level and GPBB level can be measured in the same sample or different samples.

In some embodiments, the assay comprises a substrate with binding reagents can bind to both GPBB and troponin (e.g., troponin T or troponin I), thus permitting simultaneous detection of GPBB and troponin. For example, the substrate composition can comprise a GPBB detection region and a troponin detection region, wherein the GPBB detection region can partially overlap, fully overlap, or minimally overlap with the troponin detection region. In one of these embodiments, the substrate composition can comprise a first binding agent specific for GPBB, and a second binding agent specific for troponin. Binding agents specific for GPBB can comprise, for example, an antibody to GPBB. Binding agents specific for troponin can comprise, for example, an antibody to troponin. Antibodies to GPBB, troponin T, or troponin I are commercially available. Non-limiting examples of antibodies to GPBB are disclosed in WO2008064903A2 and US20040023309, the content of each of which is incorporated by reference for its teaching on antibodies to GPBB. Non-limiting examples of antibodies to troponin T are disclosed in U.S. Pat. No. 6,376,206, US20110129818, EP1979748A1, U.S. Pat. No. 6,174,686, the content of each of which is incorporated by reference for its teaching on antibodies to troponin T. Non-limiting examples of antibodies to troponin I are disclosed in U.S. Pat. No. 6,174,686, U.S. Pat. No. 7,479,278, U.S. Pat. No. 8,030,026, US20110256640, and EP1979748A1, the content of each of which is incorporated by reference for its teaching on antibodies to troponin I.

In some embodiments, the substrate of the assay is in the format of a dipstick, a microfluidic chip or a cartridge. In some embodiments, the dipstick is a lateral flaw immunoassay test strip. Any substrate can be used, including but not limited to, nitrocellulose, solid organic polymers, such as polystyrene, or laminated dipsticks such as described in U.S. Pat. Nos. 5,550,375 and 5,656,448, which is specifically incorporated herein by reference in their entirety.

The amino acid sequence of GPBB is known in the art. It is listed here for reference:

| (SEQ ID NO: 1) | |
|---|---|
| 1 | makpltdsek rkqisvrgla glgdvaevrk sfnrhlhftl vkdrnvatpr dyffalahtv |
| 61 | rdhlvgrwir tqqhyyerdp kriyylslef ymgrtlqntm vnlglqnacd eaiyqlgldl |
| 121 | eeleeieeda glgngglgrl aacfldsmat lglaaygygi ryefgifnqk ivngwqveea |
| 181 | ddwlrygnpw ekarpeymlp vhfygrveht pdgvkwldtq vvlampydtp vpgyknntvn |
| 241 | tmrlwsakap ndfklqdfnv gdyieavldr nlaenisrvl ypndnffegk elrlkqeyfv |
| 301 | vaatlqdiir rfksskfgcr dpvrtcfetf pdkvaiqlnd thpalsipel mrilvdvekv |
| 361 | dwdkaweitk ktcaytnhtv lpealerwpv smfekllprh leiiyainqr hldhvaalfp |
| 421 | gdvdrlrrms vieegdckri nmahlcvigs havngvarih seivkqsvfk dfyelepekf |
| 481 | qnktngitpr rwlllcnpgl adtivekige efltdlsqlk kllplvsdev firdvakvkq |
| 541 | enklkfsafl ekeykvkinp ssmfdvhvkr iheykrqlln clhvvtlynr ikrdpakafv |
| 601 | prtvmiggka apgyhmakli iklvtsigdv vnhdpvvgdr lkviflenyr vslaekvipa |
| 661 | adlsqqista gteasgtgnm kfmlngalti gtmdganvem aeeagaenlf ifglrvedve |
| 721 | aldrkgynar eyydhlpelk qavdqissgf fspkepdcfk divnmlmhhd rfkvfadyea |
| 781 | ymqcqaqvdq lyrnpkewtk kvirniacsg kfssdrtite yareiwgvep sdlqipppni |
| 841 | prd |

Ischemic Stroke Vs. Hemorrhagic Stroke

Strokes can be divided into two major subtypes: an ischemic stroke (i.e., AIS) or a hemorrhagic stroke. A hemorrhagic stroke is caused by a weakened blood vessel that ruptures and bleeds into the surrounding brain tissue, and it is not an ischemic condition. The ability to distinguish an AIS from a hemorrhagic stroke is very important, as they require distinctly different treatments. An antithrombotic agent is typically administered to a patient suffering from an AIS to dissolve the blood clot. However, if an antithrombotic agent is administered to a patient suffering from a hemorrhagic stroke, it would worsen the bleeding. Hence, it is critical to be able to properly diagnose whether a patient has an ischemic stroke or a hemorrhagic stroke.

Without wishing to be bound by theory, because a surge of GPBB level in the blood occurs in response to ischemia, if the only neurological condition a subject is experiencing is a hemorrhagic stroke, the GPBB level in the blood of the subject should not be expected to increase. Accordingly, one aspect of the technology disclosed herein relates to an assay and a method of distinguishing an AIS from a hemorrhagic stroke, comprising (i) measuring, in a sample obtained from a subject experiencing a stroke or stroke-like symptoms, a level of GPBB; (ii) comparing the level of GPBB with a reference level; and identifying the subject as having an AIS if the level of GPBB is at or above a normal reference level, or a hemorrhagic stroke if the level of GPBB is below the reference level.

In some embodiments, the assay and method described herein can also distinguish a TIA from a hemorrhagic stroke.

### Detection Methods

The level of GPBB can be measured by a variety of methods including, but not limited to, enzyme-linked immunosorbent assay (ELISA), surface plasmon resonance, photonic crystal-based detection, and lateral flow immunoassay (e.g., DIACORDON™ POCT lateral flow immunoassay manufactured by Diagenics). The level of GPBB can also be measured by measuring the enzyme activity of GPBB. As but one example, Rabitzch et al. describe an immunoenzymometric assay for human GPBB (Clinica Chem. 41: 966-978 (1995).

In some embodiments, the level of GPBB is measured by ELISA, also called enzyme immunoassay or EIA. GPBB-ELISA test kits are commercially available, and can be used in the present invention to measure the level of GPBB.

In one embodiment, an ELISA involving at least one antibody with specificity for the particular desired antigen (i.e. GPBB) can be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against GPBB or a fragment thereof are coated on the solid phase of multi-well plate, i.e., conjugated to a solid surface. A GPBB-horseradish peroxidase (GPBB-HRP) conjugate is used to compete with GPBB for binding sites and to produce a detectable signal. A sample (e.g., blood or serum) from a subject are then incubated together with the GPBB-HRP conjugate. After the incubation period, the wells are decanted and washed multiple times to remove unbound antigens. The wells are then incubated with a substrate, e.g., a chromopgenic or fluorogenic substrate, for the HRP enzyme. The HRP enzyme-substrate reaction produces a blue complex. In the end, a solution is added to stop the reaction, turning the solution to yellow. The intensity of solution color can be measured by an instrument (e.g., an ELISA reader). Because GPBB and GPBB-HRP compete for antibody binding sites on the well surface, the intensity of solution color is inversely proportional to the level of GPBB. Low intensity of the solution color indicates a high level of GPBB, while high intensity of the solution color indicates a low level of GPBB. Such a competitive ELSA test is specific, sensitive, reproducible and easy to operate.

There are other different forms of ELISA, which are well known to those skilled in the art. Standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference for their teachings on ELISA.

### Reference Level

In some embodiments, the reference level can be a level of GPBB in a sample (e.g., blood) of a healthy subject with no signs or symptoms of brain ischemia or myocardial ischemia. This would be a “normal” level. In some embodiments, the reference level can be a level of GPBB in a control sample, a pooled sample of control individuals, or a numeric value or range of values based on the same. In some embodiments, the reference level is 6 ng/mL of blood based on clinical data. It is also contemplated that a set of standards can be established with reference levels providing thresholds indicative of the severity of stroke or ischemic brain status.

In some embodiments, the reference level can be a level of GPBB in a sample (e.g., blood) of a subject with no signs or symptoms of brain ischemia or myocardial ischemia, but the subject is at risk of brain ischemia. In these embodiments, the reference level can be an average level of GPBB measured in multiple samples (e.g., blood) of the subject at multiple time points.

In some embodiments, the level of GPBB measured in a sample from a subject suspected of having a stroke or exhibiting stroke symptoms can be at least 5% higher than the reference level. In some embodiments, the level of GPBB measured in a sample from a subject suspected of having a stroke can be at least 10% higher than the reference level. In some embodiments, the level of GPBB measured in a sample from a subject suspected of having a stroke can be at least 20% higher than the reference level. In some embodiments, the level of GPBB measured in a sample from a subject suspected of having a stroke can be at least 40% higher than the reference level. In some embodiments, the level of GPBB measured in a sample from a subject suspected of having a stroke can be at least 100% higher than the reference level. In some embodiments, the level of GPBB measured in a sample from a subject suspected of having a stroke can be at least 200% higher than the reference level.

It should be noted that the reference level can be different, depending on factors such as gender, age, weight, and ethnicity. Thus, reference levels accounting for these and other variables can provide added accuracy for the methods described herein.

### Kits

In some embodiments, the assays described herein can be included in a kit. In some embodiments, the kit comprises a substrate composition with binding reagent(s) for GPBB and, optionally one or more additional markers as described herein. Kits as described herein can include, in addition to a substrate with binding reagents, those reagents necessary to detect bound GPBB and the additional marker(s). In some embodiments, the kit further comprises packaging materials. The kit can include written instructions for a user on how to use the assay, and optionally on how to interpret the result.

In some embodiments, the kit can be used for point-of-care testing. By way of examples only, the kit can be used at the location of a subject exhibiting stroke symptoms. The kit can also be used in an ambulance that transports the subject to a hospital or clinic. The kit can also be used in a hospital or clinic.

### Treatment

An early, rapid, and accurate diagnosis of strokes can benefit a subject suspected of having a stroke in at least the following related aspects: (1) it can reduce the rate of misdiagnosis of strokes; (2) it can limit the extent of tissue death by permitting early and proper treatment in a subject in need of treatment.

Generally, an ischemic stroke can be treated by administering an antithrombotic agent to dissolve the blood clot. Antithrombotic agents are further divided into the following three subtypes: anticoagulants, antiplatelet drugs, and thrombolytic drugs. Non-limiting examples of anticoagulants include: coumarins, heparin, warfarin, acenocoumarol, phenprocoumon, atromentin, phenindione, fondaparinux, idraparinux, direct factor Xa inhibitors, direct thrombin inhibitors, antithrombin protein therapeutics, batroxobin, and hementin. Non-limiting examples of antiplatelet drugs include: irreversible cyclooxygenase inhibitors (e.g., aspirin or triflusal), adenosine diphosphate receptor inhibitors (e.g., clopidogrel, prasugrel, ticagrelor, or ticlopidine), phosphodiesterase inhibitors (e.g., cilostazol), glycoprotein IIB/IIIA inhibitors (e.g., abciximab, eptifibatide, or tirofiban), adenosine reuptake inhibitors (e.g., dipyridamole), and thromboxane inhibitors (e.g., thromboxane synthase inhibitors or thromboxane receptor antagonists). Non-limiting examples of thrombolytic drugs include: tissue plasminogen activator t-PA (e.g., alteplase, reteplase, or tenecteplase), anistreplase, streptokinase, and urokinase. An ischemic stroke can also be treated by endovascular procedures, in which a catheter is sent to the blood flow blockage site to remove the blood clot. t-PA can be optionally administered during the endovascular procedures.

Treatment options for a hemorrhagic stroke include, but are not limited to, endovascular procedures and surgery.

Yet another aspect of the technology disclosed herein relates to stroke treatments based on the diagnosis using the assays and/or methods described herein. For example, a treatment appropriate for AIS or TIA is provided to the subject in need thereof after the diagnosis. In yet another example, after distinguishing whether a subject is having an AIS or a hemorrhagic stroke, a treatment appropriate for the diagnosed condition is provided to the subject.

A further aspect of the technology disclosed herein regards a method of classifying or labeling a drug or drug dosage form for treatment of central nervous system (CNS) indications, the method comprising classifying or labeling the drug or drug dosage form according to the GPBB level in a sample from a patient in need of the drug or drug dosage form. In this aspect, the drug is labeled or associated with a classification based upon the patient's GPBB level (optionally considering other marker(s)). In some embodiments, if the GPBB level of the patient is at or above a reference level, then the drug or drug dosage form is classified or labeled for administration for a CNS indication, and if the GPBB level is below the reference level, then the drug or drug dosage form is classified or labeled against administration. In some embodiments, the CNS indication is an AIS or TIA, and the drug or drug dosage form is antithrombotic. This classification method can, for example, aid a clinician or pharmacist in properly giving the drug or drug dosage form to the patient in need.

By monitoring the GPBB level in a subject suffering from an ischemic stroke and receiving a treatment for the stroke, the treatment can be determined to be effective if the GPBB level, measured after the administration of a therapeutic agent for ischemic stroke, is lower than a reference level set for the time point when GPBB is measured. For monitoring purposes, GPBB levels can be measured at multiple time points, for example, 2, 3, 4, 5, 6, 7, 8, 9, 10 or more. In this manner, monitoring of GPBB levels over time following or during treatment can provide a measure of the success of treatment, as it is to be expected that GPBB levels will decrease faster in patients with successful treatment relative to an untreated patient for which GPBB levels may decrease, albeit more slowly.

### Computer Systems

In some embodiments of the assays and/or methods described herein, the assay/method comprises or consists essentially of a system for determining (e.g., measuring) the level of GPBB and/or troponin as described herein and comparing it to a reference level. If the comparison system, which can be a computer implemented system, indicates that the amount of the measured GPBB and/or troponin is at or above the reference level, the subject from which the sample is collected can be identified as, e.g. having an AIS, thereby indicating a treatment regimen.

In one embodiment, provided herein is a system comprising: (a) at least one memory containing at least one computer program adapted to control the operation of the computer system to implement a method that includes (i) a determination module configured to identify and detect the level of GPBB and/or troponin in a sample obtained from a subject; (ii) a storage module configured to store output data from the determination module; (iii) a computing module adapted to identify from the output data whether the level of GPBB and/or troponin in the sample obtained from the subject is at, above or below a reference level, and (iv) a display module, and (b) at least one processor for executing the computer program.

Embodiments can be described through functional modules, which are defined by computer executable instructions recorded on computer readable media and which cause a computer to perform method steps when executed. The modules are segregated by function for the sake of clarity. However, it should be understood that the modules/systems need not correspond to discrete blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media include volatile and nonvolatile, removable and non-removable tangible media implemented in any method or technology for storage of information such as computer readable instructions, data structures, program modules or other data. Computer readable storage media include, but are not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. Computer-readable storage media do not include a signal.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments can include at minimum a determination module, a storage module, a computing module, and a display module. The functional modules can be executed on one, or multiple, computers, or by using one, or multiple, computer networks. The determination module has computer executable instructions to provide e.g., levels of compounds etc in computer readable form.

The determination module can comprise any system capable of measuring GPBB and/or troponin in a biological sample.

The information determined in the determination system can be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample names, levels of GPBB. Such information may be provided in digital form that can be transmitted and read electronically, e.g., via the Internet, on diskette, via USB (universal serial bus) or via any other suitable mode of communication.

The “computing module” can use a variety of available software programs and formats for computing the level of GPBB and/or troponin. Such algorithms are well established in the art. A skilled artisan is readily able to determine the appropriate algorithms based on the size and quality of the sample and type of data. The data analysis can be implemented in the computing module. In one embodiment, the computing module further comprises a comparison module, which compares the level of GPBB and/or troponin in a sample obtained from a subject as described herein with a reference level. In certain embodiments, the reference level can have been pre-stored in the storage module. During the comparison or matching process, the comparison module can determine whether the GPBB and/or troponin level in the sample obtained from the subject is at or above the reference level. In various embodiments, the comparison module can be configured using existing commercially-available or freely-available software for comparison purpose, and may be optimized for particular data comparisons that are conducted.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

### Subjects

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein.

Preferably, the subject is a mammal. The mammal can be a human, non-human primate, mouse, rat, dog, cat, horse, or cow, but are not limited to these examples.

In some embodiments, a subject can be one who has been previously diagnosed with or identified as suffering from or having a stroke-related condition in need of monitoring (e.g., stroke or TIA) or one or more complications related to such a condition, and optionally, have already undergone treatment for the condition or the one or more complications related to the condition.

In some embodiments, a subject can also be one who has not been previously diagnosed as having a stroke-related condition (e.g., stroke or TIA) or one or more complications related to the condition. For example, a subject can be one who exhibits one or more risk factors for a stroke-related condition or one or more complications related to the condition or a subject who does not exhibit risk factors. Risk factors for strokes can include, but are not limited to high blood pressure, atrial fibrillation, high cholesterol, diabetes, atherosclerosis, circulation problems, tobacco use, alcohol use, physical inactivity, obesity, age, gender, race, family history, previous stroke, previous TIA, fibromuscular dysplasia, and patent foramen ovale.

A “subject in need” of treatment for a stroke-related condition can be a subject having that condition, diagnosed as having that condition, or at risk of developing that condition.

### DEFINITIONS

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise. The abbreviation, “e.g.” is derived from the Latin exempli gratia, and is used herein to indicate a non-limiting example. Thus, the abbreviation “e.g.” is synonymous with the term “for example.”

The term “sample” or “biological sample” as used herein denotes a sample taken or isolated from a biological organism, e.g., a tumor sample from a subject. Exemplary biological samples include, but are not limited to, a biofluid sample; blood; serum; plasma; urine; saliva; a tumor sample; a tumor biopsy and/or tissue sample etc. The term also includes a mixture of the above-mentioned samples. The term “sample” also includes untreated or pretreated (or pre-processed) biological samples. In some embodiments, a sample can comprise one or more cells from the subject.

As used herein, the terms “treat,” “treatment,” “treating” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to alleviation of one or more symptom(s). Treatment also includes a decrease in mortality or an increase in the lifespan of a subject as compared to one not receiving the treatment.

As used herein, “stored” refers to a process for encoding information, e.g., on a storage module. Those skilled in the art can readily adopt any of the presently known methods for recording information on known media to generate manufactures comprising concentration information.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%, or ±5%. For example, about 100 means from 95 to 105.

In one respect, the present disclosure relates to the herein described compositions, methods, and respective component(s) thereof, as essential to the invention, yet open to the inclusion of unspecified elements, essential or not (“comprising”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

Some embodiments of the invention are listed in the following paragraphs:
1. An assay comprising:
   (i) measuring, in a sample obtained from a subject, a level of glycogen phosphorylase-BB (GPBB) and a level of troponin;
   (ii) comparing the level of GPBB with a first reference level and the level of troponin with a second reference level, and if the level of GPBB is at or above the first reference level and if the level of troponin is below the second reference level;
   (iii) identifying the GPBB as originating from the subject's brain.
2. The assay of paragraph 2, further comprising providing a treatment based on the level of GPBB and appropriate for an ischemic brain condition.
3. The assay of paragraph 1 or 2, wherein the sample is a blood sample.
4. The assay of any of paragraphs 1 to 3, wherein the level of GPBB is measured by an immunoassay.
5. The assay of any of paragraphs 1 to 4, wherein the level of troponin is measured by an immunoassay.
6. The assay of any of paragraphs 1 to 5, wherein the troponin is troponin T, troponin I, or a combination thereof.
7. The assay of any of paragraphs 1 to 6, wherein the subject is a human.
8. An assay comprising:
   (i) measuring, in a sample obtained from a subject with one or more stroke symptoms, a level of GPBB;
   (ii) comparing the level of GPBB with a reference level; and
   (iii) identifying a condition the subject is having as (a) an acute ischemic stroke (AIS) or a transient ischemic attack (TIA) if the level of GPBB is at or above the reference level, or (b) a non-ischemic condition mimicking AIS or TIA if the level of GPBB is below the reference level.
9. The assay of paragraph 8, further comprising providing a treatment based on the level of GPBB and appropriate for the identified condition.
10. The assay of paragraph 8 or 9, wherein the sample is a blood sample.
11. The assay of any of paragraphs 8 to 10, wherein the level of GPBB is measured by an immunoassay.
12. The assay of any of paragraphs 8 to 11, further comprising measuring a level of troponin in the sample.
13. The assay of any of paragraphs 8 to 12, wherein the subject is a human.
14. An assay comprising
   (i) measuring, in a sample obtained from a subject experiencing a stroke, a level of GPBB;
   (ii) comparing the level of GPBB with a reference level; and
   (iii) identifying the subject as having (a) an AIS if the level of GPBB is at or above the reference level or (b) a hemorrhagic stroke if the level of GPBB is below the reference level.
15. The assay of paragraph 14, further comprising providing a treatment based on the identification of the stroke as ischemic or hemorrhagic.
16. The assay of paragraph 14 or 15, further comprising, if the subject is identified as having an AIS, administering an antithrombotic agent, and if the subject is identified as having a hemorrhagic stroke, not administering an antithrombotic agent.
17. The assay of any of paragraphs 14 to 16, wherein the sample is a blood sample.
18. The assay of any of paragraphs 14 to 17, wherein the level of GPBB is measured by an immunoassay.
19. The assay of any of paragraphs 14 to 18, wherein the subject does not have myocardial infarction.
20. The assay of any of paragraphs 14 to 19, wherein the subject is a human.
21. A substrate composition comprising a first binding agent which specifically binds to GPBB and a second binding agent which specifically binds to troponin.
22. The substrate composition of paragraph 21, wherein the first binding agent comprises a first antibody to GPBB, and wherein the second binding agent comprises a second antibody to troponin.
23. The substrate composition of paragraph 21 or 22, wherein the troponin is troponin T, troponin I, or a combination thereof
24. A method of diagnosing a stroke in a subject with one or more stroke symptoms, the method comprises contacting a sample obtained from the subject with a substrate composition of any of paragraphs 21 to 23.
25. The method of paragraph 24, wherein the sample is a blood sample.
26. A method of identifying whether a subject exhibiting one or more stroke symptoms, has an AIS or a TIA, comprising
   (i) assaying a level of GPBB in a sample obtained from the subject;
   (ii) comparing the level of GPBB with a reference level; and
   (iv) identifying a condition the subject is having as (a) an AIS or a TIA if the level of GPBB is at or above the reference level, or (b) a non-ischemic condition mimicking AIS or TIA if the level of GPBB is below the reference level.
27. The method of paragraph 26, wherein the sample is a blood sample.
28. The method of paragraph 26 or 27, wherein the level of GPBB is measured by an immunoassay.
29. The method of any of paragraphs 26 to 28, further comprising assaying a level of troponin in the sample.
30. The method of paragraph 29, wherein the levels of GPBB and troponin are measured by contacting the sample with a substrate composition of any of paragraphs 21 to 23.
31. The method of any of paragraphs 26 to 30, wherein the subject is a human.
32. A method of distinguishing brain ischemia from a heart condition in a subject, comprising
   (i) assaying a level of GPBB in a sample obtained from the subject;
   (ii) assaying a level of troponin in a sample obtained from the subject;
   (iii) comparing the level of GPBB with a first reference level and the level of troponin with a second reference level, and if the level of GPBB is at or above the first reference level and if the level of troponin is below the second reference level;
   (iv) identifying the subject as having brain ischemia.
33. The method of paragraph 32, wherein the sample is a blood sample.
34. The method of paragraph 32 or 33, wherein the level of GPBB is measured by an immunoassay.
35. The method of any of paragraphs 32 to 34, wherein the level of troponin is measured by an immunoassay.
36. The method of any of paragraphs 32 to 35, wherein the troponin is troponin T, troponin I, or a combination thereof.
37. The method of paragraph 32, wherein the levels of GPBB and troponin are measured by contacting the sample with a substrate composition of any of paragraphs 21 to 23.
38. The method of any of paragraphs 32 to 37, wherein the subject is a human.
39. A method of distinguishing an ischemic stroke from a hemorrhagic stroke in a subject experiencing a stroke, comprising
   (i) assaying a level of GPBB in a sample obtained from the subject;
   (ii) comparing the level of GPBB with a reference level; and
   (iii) identifying the subject as having (a) an AIS if the level of GPBB is at or above the reference level or (b) a hemorrhagic stroke if the level of GPBB is below the reference level.
40. The method of paragraph 39, wherein the sample is a blood sample.
41. The method of 39 or 40, wherein the level of GPBB is measured by an immunoassay.
42. The method of any of paragraphs 39 to 41, wherein the subject does not have myocardial infarction.
43. The method of any of paragraphs 39 to 42, wherein the subject is a human.
44. The method of any of paragraphs 39 to 43, further comprising administering a treatment according to the type of stroke identified.
45. A method of treating a subject exhibiting one or more stroke symptoms, the method comprising:
   (i) assaying a level of GPBB in a sample obtained from the subject;
   (ii) comparing the level of GPBB with a reference level;
   (iii) identifying the subject as having an AIS if the level of GPBB is at or above the reference level, and if the subject is identified as having an AIS;
   (iv) administering a treatment for AIS to the subject.
46. The method of paragraphs 45, wherein the sample is a blood sample.
47. The method of 45 or 46, wherein the level of GPBB is measured by an immunoassay.
48. The method of any of paragraphs 45 to 47, further comprising assaying a level of troponin in the sample.
49. The method of paragraph 48, wherein the levels of GPBB and troponin are measured by contacting the sample with a substrate composition of any of paragraphs 21 to 23.
50. The method of any of paragraphs 45 to 49, wherein the subject is a human.
51. A method of selecting a patient for treatment, the method comprising:
   (i) assaying a level of GPBB in a sample obtained from the subject;
   (ii) comparing the level of GPBB with a reference level;
   (iii) determining, based on step (ii), whether the patient has an AIS or a TIA; and
   (iv) providing a treatment appropriate for the patient based on step (iii).
52. The method of paragraph 51, wherein the sample is a blood sample.
53. The method of paragraph 51 or 52, wherein the level of GPBB is measured by an immunoassay.
54. The method of any of paragraphs 51 to 53, further comprising assaying a level of troponin in the sample.
55. The method of paragraph 54, wherein the levels of GPBB and troponin are measured by contacting the sample with a substrate composition of any of paragraphs 21 to 23.
56. A method of classifying or labeling a drug or drug dosage form for treatment of central nervous system (CNS) indications, the method comprising classifying or labeling the drug or drug dosage form according to the GPBB level in a sample from a patient in need of the drug or drug dosage form, wherein the drug or dosage form is classified or labeled for administration for a CNS indication if the GPBB level of the patient is at or above a reference level and classified or labeled against administration if the patient's GPBB level is below the reference level.
57. The method of paragraph 56, wherein the CNS indication is an AIS or a TIA.
58. The method of paragraph 57, wherein the drug or drug dosage form is antithrombotic.
59. The method of any of paragraphs 56 to 58, wherein the sample is a blood sample.
60. A method of monitoring treatment progress in a subject suffering from an ischemic stroke, the method comprising:
(i) measuring at a first time point, a first level of GPBB in a first sample obtained from the subject; and
(ii) measuring at a second time point, a second level of GPBB in a second sample obtained from the subject, wherein the second time point is later than the first time point and after the administration of a therapeutic agent for ischemic stroke, and wherein if the second level of GPBB is lower than a reference value set for the second time point, then the treatment is considered to be effective.
61. The method of paragraph 60, wherein the first sample and the second sample are blood samples.
62. The method of paragraph 60 or 61, wherein the subject is a human.
63. A method of monitoring an extent of ischemic brain injury in a subject who has an AIS or a TIA, the method comprising:
   measuring, at two or more time points, levels of GPBB from samples obtained from the subject, wherein an increase in the level of GPBB indicates a likelihood of expansion of ischemic brain injury.
64. The method of paragraph 63, wherein the samples are blood samples.
65. The method of paragraph 63 or 64, wherein the subject is a human.
66. A kit comprising a substrate composition of any of paragraphs 21 to 23, and packaging materials.
67. A method of identifying whether a subject with an AIS or a TIA exhibits salvageable brain tissue after stroke, comprising,
   (i) assaying a level of GPBB in a sample obtained from the subject;
   (ii) performing neuroimaging on the subject's brain;
   (iii) identifying a size of irreversibly injured brain tissue based on the neuroimaging;
   (iv) correlating the size of irreversibly injured brain tissue with an expected GPBB level; and
   (v) identifying the subject as having salvageable brain tissue if the level of GPBB is above the expected GPBB level.
68. The method of paragraph 67, wherein the sample is a blood sample.
69. The method of paragraph 67 or 68, wherein the neuroimaging is magnetic resonance imaging or a computed tomography scan.
70. The method of any of paragraphs 67 to 69, wherein the subject is a human.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### GPBB Levels are Elevated in Acute Ischemic Stroke

Material and Methods.

49 samples were selected from a cohort of consecutive AIS patients enrolled in a prospective biomarker study. The mean age was 71 (+/−13) years. Subjects with a confirmed diagnosis of AIS by neuroimaging and with collected blood samples within 9 hours from symptom onset were included. A second sample was collected at 48 hours. Anterior and posterior circulation strokes were included but lacunar strokes were excluded. Samples were analyzed using a commercially available GPBB-ELISA test kit. The upper reference limit of the assay was 10 ng/ml.

Results.

The median NIH stroke scale (NIHSS) at admission was 12 (IR: 6-18), and the mean infarct volume at baseline (BL) was 42.54 (+/−45.54) ml. Forty-five patients (92%) had GPBB values above the cut-off at BL. The mean and median values at BL were 55.16 (SD: 38.66) ng/ml, and 51.46 (IR: 22.65-82.11) ng/ml respectively. The mean value at 48 hours was 38.60 (SD: 32.14) ng/ml, and the median was 30.57 (IR: 14.58-55.88) ng/ml. The levels of GPBB at BL were significantly higher than the levels at 48 hours (p=0.008). This study shows that a rapid surge in GPBB levels up to approximately 5 times the upper reference limit occurs within the first few hours of AIS.

### Example 2

### A New Biomarker for Diagnosis of Ischemic Stroke: Plasma GPBB

Methods.

Plasma GPBB levels were measured in 172 patients with imaging-confirmed brain infarction at two time points (within 9 hours of symptom onset and at 48 hours). The control group consisted of 97 age-matched subjects without stroke. GPBB levels were determined blind to case/control assignment using a commercially available ELISA kit (Diagenics, Germany).

Results.

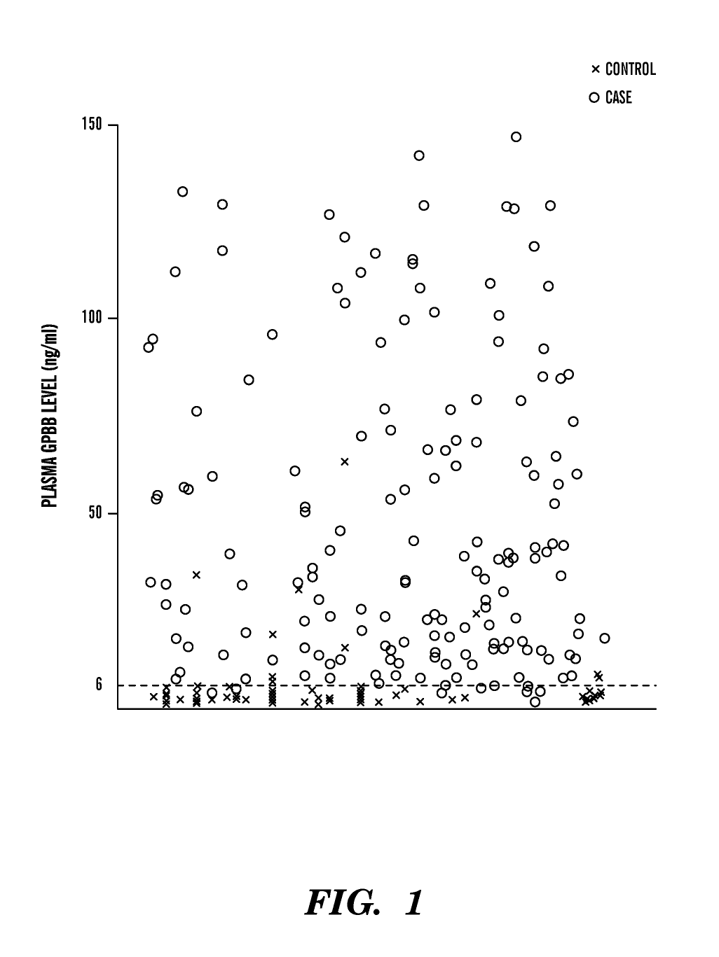

Baseline mean (±SD) GPBB level was 46.3 ng/ml (±38.6 ng/ml) in cases and 4.8 ng/ml (±8.0 ng/ml) in controls (p<0.001, FIG. 1). GPBB levels slightly decreased from baseline to 48 hours in patients with stroke (38.7±36.5 ng/ml, p=0.02). The discriminative ability of GPBB between cases and controls as measured by the area under the Receiver Operating Characteristic (ROC) curve was high (0.95, 95% CI 0.93-0.98). GPBB level corresponding to optimal operating point on the ROC curve was 6 ng/ml. Based on the 6 ng/ml cut-off, baseline plasma GPBB showed 94% sensitivity and 90% specificity for diagnosis of ischemic stroke.

TABLE 1
Yield of GPBB in TIA/minor stroke

| | GPBB positive | GPBB negative | positivity rate |
|---|---|---|---|
| TIA | 14 | 5 | 74% |
| Minor Stroke | 18 | 7 | 72% |

TABLE 2
Measurement of Troponin, in conjunction with
GPBB, to exclude diagnostic error arising from
potential leaks of GPBB from the heart

| Stroke cohort N = 175 | Troponin positive | troponin negative |
|---|---|---|
| GPBB positive | 11 | 154 |
| GPBB negative | 2 | 8 |

What is claimed is:

1-70. (canceled)

71. A method of identifying whether a subject with an acute ischemic stroke (AIS) or a transient ischemic attack (TIA) exhibits salvageable brain tissue after stroke, the method comprising:
(i) measuring a level of GPBB in a sample obtained from the subject;
(ii) performing neuroimaging on the subject's brain;
(iii) identifying a size of irreversibly injured brain tissue based on the neuroimaging;
(iv) converting the size of irreversibly injured brain tissue to an expected GPBB level; and
(v) identifying the subject as having salvageable brain tissue if the level of GPBB is above the expected GPBB level.

72. The method of claim 71, wherein the sample is a blood sample.

73. The method of claim 71, wherein the neuroimaging is magnetic resonance imaging.

74. The method of claim 71, wherein the neuroimaging is a computed tomography scan.

75. The method of claim 71, wherein the level of GPBB is measured by an immunoassay.

76. The method of claim 71, wherein the subject is a human.

77. The method of claim 71, wherein the subject does not have myocardial infarction.

78. A substrate composition comprising a first binding agent that specifically binds to GPBB and a second binding agent that specifically binds to troponin.

79. The substrate composition of claim 78, wherein the first binding agent comprises a first antibody to GPBB, and wherein the second binding agent comprises a second antibody to troponin.

80. The substrate composition of claim 78, wherein the troponin is selected from the group consisting of troponin T, troponin I, and a combination thereof.

81. A kit comprising a substrate composition having a first binding agent that specifically binds to GPBB and a second binding agent that specifically binds to troponin, and packaging materials therefor.

82. The kit of claim 81, wherein the first binding agent comprises a first antibody to GPBB, and wherein the second binding agent comprises a second antibody to troponin.

83. The kit of claim 81, wherein the troponin is selected from the group consisting of troponin T, troponin I, and a combination thereof.


SEQUENCE LISTING (1 sequence)

SEQ ID NO: 1 - protein, 843 aa, Homo sapiens
MAKPLTDSEKRKQISVRGLAGLGDVAEVRKSFNRHLHFTLVKDRNVATPRDYFFALAHTVRDHLVGRWIRTQQHYYERDPKRIYYLSLEFYMGRTLQNTMVNLGLQNACDEAIYQLGLDLEELEEIEEDAGLGNGGLGRLAACFLDSMATLGLAAYGYGIRYEFGIFNQKIVNGWQVEEADDWLRYGNPWEKARPEYMLPVHFYGRVEHTPDGVKWLDTQVVLAMPYDTPVPGYKNNTVNTMRLWSAKAPNDFKLQDFNVGDYIEAVLDRNLAENISRVLYPNDNFFEGKELRLKQEYFVVAATLQDIIRRFKSSKFGCRDPVRTCFETFPDKVAIQLNDTHPALSIPELMRILVDVEKVDWDKAWEITKKTCAYTNHTVLPEALERWPVSMFEKLLPRHLEIIYAINQRHLDHVAALFPGDVDRLRRMSVIEEGDCKRINMAHLCVIGSHAVNGVARIHSEIVKQSVFKDFYELEPEKFQNKTNGITPRRWLLLCNPGLADTIVEKIGEEFLTDLSQLKKLLPLVSDEVFIRDVAKVKQENKLKFSAFLEKEYKVKINPSSMFDVHVKRIHEYKRQLLNCLHVVTLYNRIKRDPAKAFVPRTVMIGGKAAPGYHMAKLIIKLVTSIGDVVNHDPVVGDRLKVIFLENYRVSLAEKVIPAADLSQQISTAGTEASGTGNMKFMLNGALTIGTMDGANVEMAEEAGAENLFIFGLRVEDVEALDRKGYNAREYYDHLPELKQAVDQISSGFFSPKEPDCFKDIVNMLMHHDRFKVFADYEAYMQCQAQVDQLYRNPKEWTKKVIRNIACSGKFSSDRTITEYAREIWGVEPSDLQIPPPNIPRD